(19) United States
(12) Patent Application Publication
(10) Pub. No.: US 2023/0201240 A1
(43) Pub. Date: Jun. 29, 2023

(54) TREATMENT OF ADVANCED METASTATIC CANCER

(71) Applicant: Can-Fite Biopharma Ltd., Petah Tikva (IL)

(72) Inventor: Pnina FISHMAN, Herzliya (IL)

(73) Assignee: Can-Fite Biopharma Ltd., Petah Tikva (IL)

(21) Appl. No.: 18/147,285

(22) Filed: Dec. 28, 2022

Related U.S. Application Data
(60) Provisional application No. 63/266,140, filed on Dec. 29, 2021.

(51) Int. Cl. A61K 31/7076; A61K 45/06; A61K 31/4745; A61P 35/04; A61P 1/16

(52) U.S. Cl. CPC A61K 31/7076; A61K 45/06; A61K 31/4745; A61P 35/04; A61P 1/16

(57) ABSTRACT

The present invention concerns an A₃ adenosine receptor (A₃AR) ligand for use in the treatment of an advanced solid tumor, e.g., hepatocellular carcinoma (HCC) in a mammalian subject.

### FIELD OF THE INVENTION

This invention relates to treatment of advanced metastatic cancer, in particular, advanced hepatocellular carcinoma, comprising administration of an A3AR ligand.

### BACKGROUND OF THE INVENTION

Primary liver cancer, and specifically hepatocellular carcinoma (HCC), is a major global health problem due to its incidence, associated mortality, and lack of effective treatment modalities, particularly for patients with moderate or advanced hepatic dysfunction.

Patients with advanced HCC and Child-Pugh B (CPB) cirrhosis have a borderline liver function, and therefore the benefit of any therapy might be offset by the decline in their liver function. The only curative option for these patients involves successful downstaging and liver transplantation. However, this approach is appropriate only for a minority of patients and is further limited by the restricted number of livers available for transplantation [Granito A., and Bolondi L. Lancet Oncol. 2017; 18: e101-e112]. The most common treatment for HCC and CPB is the multikinase inhibitor sorafenib, which is approved by the US Food and Drug Administration (FDA) for advanced HCC regardless of liver function.

CPB patients are generally excluded from clinical studies due to their poor prognosis and low expected response rate [Llovet J. M., et al. J. Natl. Cancer Inst. 2008; 100:698-711]. Clearly, therapies for HCC and CPB cirrhosis are still needed.

The Gi protein-coupled A3 adenosine receptor (A3AR) is overexpressed in different types of solid tumors, including melanoma, breast, colon, and prostate cancer, and HCC, whereas low receptor expression is found in adjacent normal tissues [Bar-Yehuda S., et al. Int. J. Oncol. 2008; 33:287-295].

Namodenoson, generically known as Cl-IB-MECA, is a highly selective orally bioavailable A3AR agonist that induces an apoptotic effect towards HCC in syngeneic orthotopic and xenograft experimental animal models [Cohen S., et al., J. Cell Physiol. 2011; 226:2438-2447].

In an open-label phase I/II trial, the safety and efficacy of namodenoson were assessed in patients with advanced unresectable HCC, 67% of whom failed prior sorafenib treatment. Median overall survival (OS) was 7.8 months for the whole study population, and for CPB patients (28%), it was 8.1 months. Namodenoson was safe and well-tolerated, and a direct correlation between A3AR expression levels at baseline and tumor response to namodenoson was found [Stemmer S. M., et al. Oncologist. 2013; 18:25-26].

### SUMMARY OF THE INVENTION

In a first of its aspects, the present invention provides a method of treating an advanced solid tumor, said method comprising administering to a mammalian subject in need thereof an A₃ adenosine receptor (A₃AR) ligand or a pharmaceutical composition comprising said A₃AR ligand.

In another aspect, the present invention provides a pharmaceutical composition comprising an A₃AR ligand, and a pharmaceutically acceptable carrier or diluent, wherein said pharmaceutical composition is for treating an advanced solid tumor in a mammalian subject.

In an embodiment, said advanced solid tumor is advanced hepatocellular carcinoma.

In an embodiment, said advanced solid tumor is metastatic hepatocellular carcinoma.

In an embodiment, said subject has advanced hepatocellular carcinoma with Child-Pugh B (CPB) cirrhosis score of 7 (CPB7), Child-Pugh B (CPB) cirrhosis score of 8 (CPB8), or Child-Pugh B (CPB) cirrhosis score of 9 (CPB9).

In an embodiment, said A₃AR ligand is an A₃AR agonist or an A₃AR allosteric modulator.

In some embodiments, said A₃AR agonist is selected from the group consisting of N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA, namodenoson).

In some embodiments, said A₃AR allosteric modulator is selected from the group consisting of:
N-(3,4-Dichloro-phenyl)-2-cyclopentyl-1H-imidazo[4,5-c]quinolin-4-amine;
N-(3,4-Dichloro-phenyl)-2-cycloheptyl-1H-imidazo[4,5-c]quinolin-4-amine;
N-(3,4-Dichloro-phenyl)-2-cyclobutyl-1H-imidazo[4,5-c]quinolin-4-amine; and
N-(3,4-Dichloro-phenyl)-2-cyclohexyl-1H-imidazo[4,5-c]quinolin-4-amine.

In an embodiment, the treatment further comprises administration of an additional therapeutic agent.

In some embodiments, said additional therapeutic agent is an anti-cancer drug, e.g., a monoclonal antibody and/or a multi-kinase inhibitor.

In some embodiments, said A₃AR ligand is administered once daily, twice daily, or thrice daily.

In an embodiment, said A₃AR ligand is administered every 12 hours throughout the treatment period.

In an embodiment, said A₃AR ligand is administered in a continuous manner.

In an embodiment, said treatment period is divided into cycles (e.g., 4-week cycles).

In an embodiment, said mammalian subject is a human subject.

In some embodiments, said A₃AR ligand is administered at an amount of 50 μg/kg-10 mg/kg body weight, preferably 100 μg/kg-5 mg/Kg body weight, or 200 μg/kg-1 mg/Kg body weight.

In a specific embodiment, said A₃AR ligand is Cl-IB-MECA which is administered orally in a dose of 1-50 mg, preferably 5-30 mg twice daily.

In a specific embodiment, said subject received the A₃AR ligand as a second-line therapy.

In a specific embodiment, said administration is for a treatment period of at least 9 months, at least 10 months, at least one year, at least 2 years, at least 3 years, at least 4 years or at least 5 years.

In another aspect, the present invention provides a method of increasing overall survival of subjects with advanced HCC and a CPB7 score, said method comprises administering an A₃AR ligand (e.g., Cl-IB-MECA) to said subject.

In a specific embodiment, said increase in overall survival is measured after a treatment period of 9 months, 10 months, 12 months or more, and wherein said treatment comprises administration of the A₃AR ligand (e.g., Cl-IB-MECA) orally in a dose of 1-50 mg, preferably 5-30 mg twice daily.

In another aspect, the present invention provides a kit comprising:
(a) a pharmaceutical composition comprising an A₃AR ligand as described above;
(b) instructions for administration of the pharmaceutical composition for the treatment of a subject with an advanced solid tumor.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a graph showing comparison of 12-month Overall Survival (OS) Rate in Patients with Child-Pugh Score 7 (Namodenoson 25 mg BID vs placebo).

### DETAILED DESCRIPTION OF EXEMPLARY EMBODIMENTS

The present invention is based on the surprising finding that a patient with advanced HCC with CPB that was treated with namodenoson (Cl-IB-MECA) showed unprecedent results and complete remission of the cancer after 5 years of treatment. The patient was included in a randomized, placebo-controlled, phase II study evaluating the efficacy/safety of namodenoson versus placebo in advanced HCC CPB patients. Moreover, the phase II study showed a surprising efficacy of namodenoson in increasing 12-months overall survival rate of the treated patients.

The invention is described in the following detailed description with reference to therapeutic methods for the treatment of advanced solid tumors, in particular, HCC with CPB involving administration of an A₃AR ligand to a subject in need of same.

As used in the specification and claims, the forms “a”, “an” and “the” include singular as well as plural references unless the context clearly dictates otherwise. For example, the term “an A3AR ligand” includes one or more ligands.

Further, as used herein, the term “comprising” is intended to mean that the method or composition includes the recited elements but does not exclude others. Similarly, “consisting essentially of” is used to define methods and compositions that include the recited elements but exclude other elements that may have an essential significant therapeutic activity towards joint inflammation. For example, a composition consisting essentially of an A₃AR ligand will not include or include only insignificant amounts (amounts that will have an insignificant effect on joint inflammation) of other active ingredients that have such an activity. Also, a composition consisting essentially of the A₃AR ligand as defined herein would not exclude trace contaminants from the isolation and purification method, pharmaceutically acceptable carriers, such as phosphate buffered saline, excipients, preservatives, and the like. “Consisting of” shall mean excluding more than trace elements of other elements. Embodiments defined by each of these transition terms are within the scope of this invention.

Further, all numerical values, e.g., concentration or dose or ranges thereof, are approximations which are varied (+) or (−) by up to 20%, at times by up to 10% of the stated values. It is to be understood, even if not always explicitly stated that all numerical designations are preceded by the term “about”. It also is to be understood, although not always explicitly stated, that the reagents described herein are merely exemplary and that equivalents of such are known in the art.

There is provided by the present invention a method of treating an advanced solid tumor said method comprising administering to a mammalian subject in need thereof an A₃ adenosine receptor (A₃AR) ligand or a pharmaceutical composition comprising said A₃AR ligand.

In a specific embodiment, the advanced solid tumor is advanced hepatocellular carcinoma (HCC).

In the context of the present invention the term “treatment” comprises treating an advanced solid tumor, e.g., advanced HCC by administering a therapeutically effective amount of an A₃AR ligand to achieve a desired therapeutic effect. The desired therapeutic effect may include, without being limited thereto, complete, or partial reversal of disease symptoms, clearing of the cancer lesions, increasing survival, achieving disappearance of ascites, normal liver function, and the disappearance of peritoneal carcinomatosis.

As used herein the term “advanced solid tumor” refers to a malignant solid neoplasm that has spread extensively to other anatomic sites or is no longer responding to treatment. Non-limiting examples of malignant solid neoplasms include carcinomas (e.g., adenocarcinoma, breast carcinoma, ovarian carcinoma, non-small cell lung cancer, bladder cancer, prostate cancer, colon cancer, hepatocellular carcinoma, squamous cell carcinoma or glioma), and sarcomas (e.g., bone, tendon, cartilage, muscle, or fat sarcomas).

As used herein the term “advanced hepatocellular carcinoma” refers to an advanced (metastatic) liver cancer which has spread either to the lymph nodes or to other organs. At such a stage the cancer is widespread and generally cannot be removed by surgery.

In an embodiment, the present invention concerns patients with hepatocellular carcinoma which is associated with cirrhosis. The cirrhotic state of the liver can be assessed using the Child-Pugh scoring system (also known as the Child-Pugh-Turcotte score) that was designed to predict mortality in cirrhosis patients. This scoring system breaks down patients into three categories: A—good hepatic function, B—moderately impaired hepatic function, and C—advanced hepatic dysfunction. The scoring system uses five clinical and laboratory criteria to categorize patients: serum bilirubin, serum albumin, ascites, neurological disorder (encephalopathy), and prothrombin time, each of these criteria being defined using a numerical value which together define the severity of cirrhosis:
Child-Pugh A: 5 to 6 points
Child-Pugh B: 7 to 9 points
Child-Pugh C: 10 to 15 points

In accordance with the invention, the subject may have advanced hepatocellular carcinoma with Child-Pugh B cirrhosis score of 7 to 9 points, namely Child-Pugh B (CPB) cirrhosis score of 7 (CPB7), Child-Pugh B (CPB) cirrhosis score of 8 (CPB8), or Child-Pugh B (CPB) cirrhosis score of 9 (CPB9).

In accordance with one embodiment of the invention the subjects are treated with the A₃AR ligand as a second-line therapy, namely the subjects were previously treated with another anti-cancer drug and the therapy failed. In other words, in some embodiments, the subjects in accordance with the present invention are patients with advanced HCC with CPS7-CPS9 score who failed other treatment regimens.

In an embodiment, the present invention provides a method of increasing overall survival of patients with advanced HCC with a CPB7 score.

In a specific embodiment, said increase is a 12-months or more increase in overall survival.

As used herein, the term “an A3 adenosine receptor (A3AR) ligand” encompasses A₃AR agonists as well as A₃AR allosteric modulators.

A₃AR agonists are known in the art and are readily available. Generally, an A₃AR agonist is any compound that is capable of specifically binding to the adenosine A₃ receptor (“A₃R”), thereby fully or partially activating said receptor thereby yielding a therapeutic effect (e.g., an anti-arthritic effect). The A₃AR agonist is thus a molecule that exerts its prime effect through the binding and activation of the A₃AR. This means that at the doses it is being administered it essentially binds to and activates only the A₃R.

In an embodiment, the A₃AR agonist has a binding affinity (Kᵢ) to the human A₃AR of less than 1000 nM, desirably less than 500 nM, advantageously less 200 nM and even less than 100 nM, typically less than 50 nM, preferably less than 20 nM, more preferably less than 10 nM and ideally less than 5 nM. The lower the Kᵢ, the lower the dose of the A₃AR agonist (that may be used) that will be effective in activating the A₃R and thus achieving a therapeutic effect.

It should be noted that some A₃AR agonists can also interact with and activate other receptors with lower affinities (namely a higher Ki). A molecule will be considered an A₃AR agonist in the context of the invention (namely a molecule that exerts its prime effect through the binding and activation A₃R) if its affinity to the A₃R is at least 3 times (i.e., its Ki to the A₃R is at least 3 times lower), preferably 10 times, desirably 20 times and most preferably at least 50 times larger than the affinity to any other of the adenosine receptors.

The affinity of A₃AR agonists to the human A₃R as well as its relative affinity to the other human adenosine receptors can be determined by various assays, such as a binding assay. Examples of binding assays include providing membranes or cells having the receptor and measuring the ability of the A₃AR agonist to displace a bound radioactive agonist; utilizing cells that display the respective human adenosine receptor and measuring, in a functional assay, the ability of the A₃AR agonist to activate or deactivate downstream signaling events such as the effect on adenylate cyclase measured through increase or decrease of the cAMP level; etc. Clearly, if the administered level of an A₃AR agonist is increased such that its blood level reaches a level approaching that of the Ki of the other adenosine receptors, activation of these receptors may occur following such administration, in addition to activation of the A₃R. An A₃AR agonist is thus preferably administered at a dose such that the blood level that will be attained will give rise to essentially only A₃R activation.

The characteristic of some adenosine A₃AR agonists and methods of their preparation are described in detail in, inter alia, U.S. Pat. Nos. 5,688,774; 5,773,423; 5,573,772; 5,443,836; 6,048,865; WO 95/02604; WO 99/20284; WO 99/06053; WO 97/27173 and WO 01/19360, all of which are incorporated herein by reference.

The following examples are specified in U.S. Pat. No. 5,688,774 at column 4, lines 67-column 6, line 16; column 5, lines 40-45; column 6, lines 21-42; column 7, lines 1-11; column 7, lines 34-36; and column 7, lines 60-61:
N⁶-(3-iodobenzyl)-9-methyladenine;
N⁶-(3-iodobenzyl)-9-hydroxyethyladenine;
R—N⁶-(3-iodobenzyl)-9-(2,3-dihydroxypropyl)adenine;
S—N⁶-(3-iodobenzyl)-9-(2,3-dihydroxypropyl)adenine;
N⁶-(3-iodobenzyladenin-9-yl)acetic acid;
N⁶-(3-iodobenzyl)-9-(3-cyanopropyl)adenine;
2-chloro-N⁶-(3-iodobenzyl)-9-methyladenine;
2-amino-N⁶-(3-iodobenzyl)-9-methyladenine;
2-hydrazido-N⁶-(3-iodobenzyl)-9-methyladenine;
N⁶-(3-iodobenzyl)-2-methylamino-9-methyladenine;
2-dimethylamino-N⁶-(3-iodobenzyl)-9-methyladenine;
N⁶-(3-iodobenzyl)-9-methyl-2-propylaminoadenine;
2-hexylamino-N⁶-(3-iodobenzyl)-9-methyladenine;
N⁶-(3-iodobenzyl)-2-methoxy-9-methyladenine;
N⁶-(3-iodobenzyl)-9-methyl-2-methylthioadenine;
N⁶-(3-iodobenzyl)-9-methyl-2-(4-pyridylthio)adenine;
(1S, 2R, 3S, 4R)-4-(6-amino-2-phenylethylamino-9H-purin-9-yl)cyclopentane-1,2,3-triol;
(1S, 2R, 3S, 4R)-4-(6-amino-2-chloro-9H-purin-9-yl) cyclopentane-1,2,3-triol;
(±)-9-[2α,3α-dihydroxy-4β-(N-methylcarbamoyl)cyclopent-10-yl)]-N⁶-(3-iodobenzyl)-adenine;
2-chloro-9-(2′-amino-2′,3′-dideoxy-β-D-5′-methyl-arabino-furonamido)-N⁶-(3-iodobenzyl)adenine;
2-chloro-9-(2′,3′-dideoxy-2′-fluoro-β-D-5′-methyl-arabino furonamido)-N⁶-(3-iodobenzyl)adenine;
9-(2-acetyl-3-deoxy-β-D-5-methyl-ribofuronamido)-2-chloro-N⁶-(3-iodobenzyl)adenine;
2-chloro-9-(3-deoxy-2-methanesulfonyl-β-D-5-methyl-ribofuronamido)-N⁶-(3-iodobenzyl)adenine;
2-chloro-9-(3-deoxy-β-D-5-methyl-ribofuronamido)-N⁶-(3-iodobenzyl)adenine;
2-chloro-9-(3,5-1,1,3,3-tetraisopropyldisiloxyl-β-D-5-ribofuranosyl)-N⁶-(3-iodobenzyl)adenine;
2-chloro-9-(2′,3′-O-thiocarbonyl-3-D-5-methyl-ribofuronamido)-N⁶-(3-iodobenzyl)adenine;
9-(2-phenoxythiocarbonyl-3-deoxy-β-D-5-methyl-ribofuronamido)-2-chloro-N⁶-(3-iodobenzyl)adenine;
1-(6-benzylamino-9H-purin-9-yl)-1-deoxy-N,4-dimethyl-β-D-ribofuranosiduronamide;
2-chloro-9-(2,3-dideoxy-3-D-5-methyl-ribofuronamido)-N⁶ benzyladenine;
2-chloro-9-(2′-azido-2′,3′-dideoxy-β-D-5′-methyl-arabino-furonamido)-N⁶-benzyladenine;
2-chloro-9-(β-D-erythrofuranoside)-N⁶-(3-iodobenzyl)adenine;
N⁶-(benzodioxanemethyl)adenosine;
1-(6-furfurylamino-9H-purin-9-yl)-1-deoxy-N-methyl-β-D-ribofuranosiduronamide;
N⁶-[3-(L-prolylamino)benzyl]adenosine-5′-N-methyluronamide;
N⁶-[3-(β-alanylamino)benzyl]adenosine-5′-N-methyluronamide;
N⁶-[3-(N-T-Boc-β-alanylamino)benzyl]adenosine-5′-N-methyluronamide
6-(N′-phenylhydrazinyl)purine-9-β-ribofuranoside-5′-N-methyluronamide;
6-(O-phenylhydroxylamino)purine-9-β-ribofuranoside-5′-N-methyluronamide;
9-(β-D-2′,3′-dideoxyerythrofuranosyl)-N⁶-[(3-β-alanylamino)benzyl]adenosine;
9-(D-D-erythrofuranoside)-2-methylamino-N⁶-(3-iodobenzyl)adenine;
2-chloro-N-(3-iodobenzyl)-9-(2-tetrahydrofuryl)-9H-purin-6-amine;
2-chloro-(2′-deoxy-6′-thio-L-arabinosyl)adenine; and
2-chloro-(6′-thio-L-arabinosyl)adenine.

In U.S. Pat. No. 5,773,423 at column 6, line 39, to column 7, line 14, specifically disclosed are compounds which include the formula:

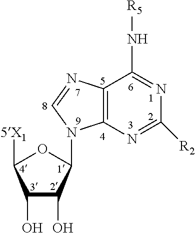

wherein

X₁ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl;

R₂ is selected from the group consisting of hydrogen, halo, C₁-C₁₀ alkyoxy, amino, C₂-C₁₀ alkenyl, and C₂-C₁₀ alkynyl; and

R₅ is selected from the group consisting of R- and S-1-phenylethyl, an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo.

More specific compounds include those of the above formula wherein R^{a} and R^{b} may be the same or different and are selected from the group consisting of hydrogen and C₁-C₁₀ alkyl, particularly when R₂ is hydrogen or halo, especially hydrogen.

Additional specific compounds are those compounds wherein R^{a} is hydrogen and R₂ is hydrogen, particularly when R₅ is unsubstituted benzyl.

More specific compounds are such compounds wherein R^{b} is a C₁-C₁₀ alkyl or C₃-C₁₀ cycloalkyl, particularly a C₁-C₁₀ alkyl, and more particularly methyl.

Especially specific are those compounds where R^{a} is hydrogen, R^{b} is C₁-C₁₀ alkyl or C₃-C₁₀ cycloalkyl, and R₅ is R- or S-1-phenylethyl or a benzyl substituted in one or more positions with a substituent selected from the group consisting of halo, amino, acetamido, C₁-C₁₀ haloalkyl, and sulfo, where the sulfo derivative is a salt, such as a triethylammonium salt.

An example of an especially preferred compound disclosed in U.S. Pat. No. 5,773,423 is IB-MECA. In addition, those compounds in which R₂ is a C₂-C₁₀ alkenylene of the formula R^{d}—C═C— where R^{d} is a C₁-C₈ alkyl are particularly noted in this publication. Also specific are those compounds wherein R₂ is other than hydrogen, particularly those wherein R₂ is halo, C₁-C₁₀ alkylamino, or C₁-C₁₀ alkylthio, and, more preferably, when additionally R^{a} is hydrogen, R^{b} is a C₁-C₁₀ alkyl, and/or R₅ is a substituted benzyl.

Such specifically disclosed compounds include 2-chloro-N⁶-(3-iodobenzyl)-9-[5-(methylamido)-β-D-ribofuranosyl]-adenine, N⁶-(3-iodobenzyl)-2-methylamino-9-[5-(methylamido)-β-D-ribofuranosyl]-adenine, and N⁶-(3-iodobenzyl)-2-methylthio-9-[5-(methylamido)-β-D-ribofuranosyl]-adenine.

Further U.S. Pat. No. 5,773,423 discloses at column 7, line 60, through column 8, line 6, A₃AR agonists as modified xanthine-7-ribosides having the formula:

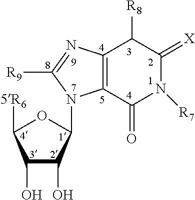

wherein

X is O;

R₆ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl;

R₇ and R₈ may be the same or different and are selected from the group consisting of C₁-C₁₀ alkyl, R- and S-1-phenylethyl, an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo; and

R₉ is selected from the group consisting of halo, benzyl, phenyl, and C₃-C₁₀ cycloalkyl.

WO 99/06053 discloses in examples 19-33 compounds selected from:
N⁶-(4-biphenyl-carbonylamino)-adenosine-5′-N-ethyluronamide;
N⁶-(2,4-dichlorobenzyl-carbonylamino)-adenosine-5′-N-ethyluronamide;
N⁶-(4-methoxyphenyl-carbonylamino)-adenosine-5′-N-ethyluronamide;
N⁶-(4-chlorophenyl-carbonylamino)-adenosine-5′-N-ethyluronamide;
N⁶-(phenyl-carbonylamino)-adenosine-5′-N-ethyluronamide;
N⁶-(benzylcarbamoylamino)-adenosine-5′-N-ethyluronamide;
N⁶-(4-sulfonamido-phenylcarbamoyl)-adenosine-5′-N-ethyluronamide;
N⁶-(4-acetyl-phenylcarbamoyl)-adenosine-5′-N-ethyluronamide;
N⁶—((R)-α-phenylethylcarbamoyl)-adenosine-5′-N-ethyluronamide;
N⁶—((S)-α-phenylethylcarbamoyl)-adenosine-5′-N-ethyluronamide;
N⁶-(5-methyl-isoxazol-3-yl-carbamoyl)-adenosine-5′-N-ethyluronamide;
N⁶-(1,3,4-thiadiazol-2-yl-carbamoyl)-adenosine-5′-N-ethyluronamide;
N⁶-(4-n-propoxy-phenylcarbamoyl)-adenosine-5′-N-ethyluronamide;
N⁶-bis-(4-nitrophenylcarbamoyl)-adenosine-5′-N-ethyluronamide; and
N⁶-bis-(5-chloro-pyridin-2-yl-carbamoyl)-adenosine-5′-N-ethyluronamide.

According to one embodiment of the invention, the A₃AR agonist is a compound that exerts its prime effect through the binding and activation of the adenosine A₃AR and is a purine derivative falling within the scope of the general formula (I):

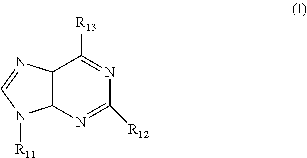

wherein,
R₁₁ represents an alkyl, hydroxyalkyl, carboxyalkyl or cyanoalkyl or a group of the following general formula (II):

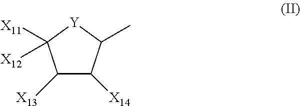

in which:
Y represents oxygen, sulfur or CH₂;
X₁₁ represents H, alkyl, R^{e}R^{f}NC(═O)— or HOR^{g}—, wherein
   R^{e} and R^{f} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
   R^{g} is selected from the group consisting of alkyl, amino, haloalkyl, aminoalkyl, BOC-aminoalkyl, and cycloalkyl;
X₁₂ is H, hydroxyl, alkylamino, alkylamido or hydroxyalkyl;
X₁₃ and X₁₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₁₃ and X₁₄ are oxygens connected to >C═S to form a 5-membered ring, or X₁₂ and X₁₃ form the ring of formula (III):

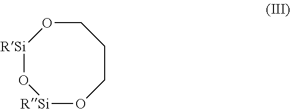

where R′ and R″ represent independently an alkyl group;
R₁₂ is selected from the group consisting of hydrogen, halo, alkylether, amino, hydrazido, alkylamino, alkoxy, thioalkoxy, pyridylthio, alkenyl; alkynyl, thio, and alkylthio; and
R₁₃ is a group of the formula —NR₁₅R₁₆ wherein
R₁₅ is a hydrogen atom or a group selected from alkyl, substituted alkyl or aryl-NH—C(Z)—, with Z being O, S, or NR^{a} with R^{e} having the above meanings; wherein when R₁₅ is hydrogen than
R₁₆ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₁₆ is a group of the following formula:

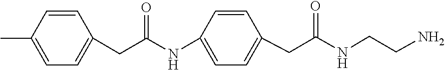 [SMILES: Cc1ccc(CC(=O)Nc2ccc(CC(=O)NCCN)cc2)cc1]

or when R₁₅ is an alkyl or aryl-NH—C(Z)—, then, R₁₆ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine; or a physiologically acceptable salt of the above compound.

According to one preferred embodiment, the A₃AR agonist is a nucleoside derivative of the general formula (IV):

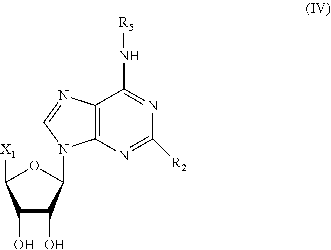

wherein X₁, R₂′ and R₅ are as defined above, and physiologically acceptable salts of said compound.

The non-cyclic carbohydrate groups (e.g., alkyl, alkenyl, alkynyl, alkoxy, aralkyl, alkaryl, alkylamine, etc) forming part of the substituent of the compounds of the present invention are either branched or unbranched, preferably containing from one or two to twelve carbon atoms.

A specific group of A3AR agonists are the N⁶-benzyladenosine-5′-uronamide derivatives. Some preferred N⁶-benzyladenosine-5′-uronamide derivatives are N⁶-2-(4-aminophenyl)ethyladenosine (APNEA), N⁶-(4-amino-3-iodobenzyl) adenosine-5′-(N-methyluronamide) (AB-MECA) and 1-deoxy-1-{6-[({3-iodophenyl} methyl)amino]-9H-purine-9-yl}-N-methyl-β-D-ribofuranuronamide (IB-MECA) and 2-chloro-N⁶-(3-iodobenzyl)adenosine-5′-N-methlyuronamide (Cl-IB-MECA).

According to another embodiment, the A₃AR agonist may be an oxide derivative of adenosine, such as N⁶-benzyladenosine-5′-N-alkyluronamide-N¹-oxide or N⁶-benzyladenosine-5′-N-dialkyluronamide-N¹-oxide, wherein the 2-purine position may be substituted with an alkoxy, amino, alkenyl, alkynyl or halogen.

When referring to an “A₃AR allosteric modulator” or “A₃ARM” it is to be understood as referring to the positive regulation, activation or increase of the receptor activity by binding of the allosteric modulator at the receptor's allosteric site which may be different from the binding site of the endogenous ligand or agonist thereof.

In one example, “modulation” denotes an effect of the A₃AR ligand on the receptor exhibited by an increase of at least 15% in the efficacy of the A₃ adenosine receptor by binding of the compound to the allosteric site of the receptor and/or by a decrease in dissociation rate of adenosine or an A₃AR agonist to the orthosteric binding site.

In one example, the modulation is by an A₃AR allosteric modulator (A₃ARAM) that is an imidazoquinoline derivative.

In one example, the A₃ARAM, or imidazoquinoline derivative has the following general formula (V):

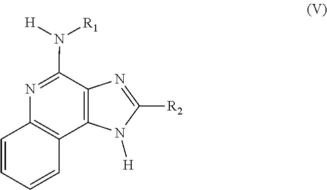

wherein:
R₁ represents an aryl or alkaryl being optionally substituted at the aromatic ring with one or more substituents selected from the group consisting of C₁-C₁₀ alkyl, halo, C₁-C₁₀ alkanol, hydroxyl, C₁-C₁₀ acyl, C₁-C₁₀ alkoxyl; C₁-C₁₀-alkoxycarbony, C₁-C₁₀ alkoxylalkyl; C₁-C₁₀ thioalkoxy; C₁-C₁₀ alkylether, amino, hydrazido, C₁-C₁₀ alkylamino, pyridylthio, C₂-C₁₀ alkenyl; C₂-C₁₀ alkynyl, thio, C₁-C₁₀ alkylthio, acetoamido, sulfonic acid; or said substituents can form together a cycloalkyl or cycloalkenyl fused to said aryl, the cycloalkyl or cycloalkenyl optionally comprising one or more heteroatoms; provided that said aryl is not an unsubstituted phenyl group;
R₂ represents hydrogen or a substituent selected from the group consisting of C₁-C₁₀ alkyl, C₂-C₁₀ alkenyl; C₂-C₁₀ alkynyl, C₄-C₁₀ cycloalkyl, C₄-C₁₀ cycloalkenyl, a five to seven membered heterocyclic aromatic ring, C₅-C₁₅ fused cycloalkyl, bicyclic aromatic or heteroaromatic rings; C₁-C₁₀ alkylether, amino, hydrazido, C₁-C₁₀ alkylamino, C₁-C₁₀ alkoxy, C₁-C₁₀-alkoxycarbony, C₁-C₁₀ alkanol, C₁-C₁₀ acyl, C₁-C₁₀ thioalkoxy, pyridylthio, thio, and C₁-C₁₀ alkylthio, acetoamido and sulfonic acid;

and pharmaceutically acceptable salts thereof.

According to some embodiments, the R₁ substituent in the A₃ARAM has the following general formula (VI):

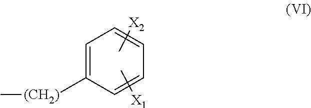

wherein n is 0 or an integer selected from 1-5; preferably, n is 0, 1 or 2; and
X₁ and X₂ which may be the same or different, are selected from hydrogen halogen, alkyl, alkanol or alkoxy, indanyl, pyrroline provided that when said n is 0, X₁ and X₂ are not hydrogen.

In yet some further examples, R₁ in A₃ARAM is a substituent having the above formula (VI), wherein X₁ or X₂, which may be the same or different, are selected from hydrogen, chloro, methoxy, methanol or a substituent having the formulae (VIa) or (VIb):

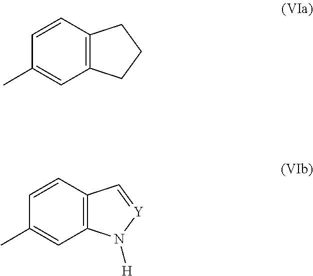

wherein Y is selected from N or CH.

In some yet further examples R₂ in A₃ARAM is selected from H, C₁₋₁₀ alkyl, C₄₋₁₀ cycloalkyl, the alkyl chain may be a straight or branched or form a four to seven membered cycloalkyl ring.

In one example, R₂ in A₃ARAM is selected from a five to seven membered heterocyclic aromatic ring.

In some examples, R₂ substituents in A₃ARAM are selected from H, n-pentyl, or a five membered heterocyclic aromatic ring having the following formula (VII):

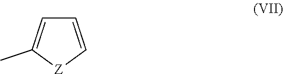

wherein Z is selected from O, S or NH, preferably O.

In accordance with one example, R₂ in the A₃ARAM comprises one or more fused rings, particularly so as to form bicyclic substituents.

Non-limiting examples of bicyclic compounds which may be used to form the substituents in the context of the A₃ARAM comprise bicyclo[2.2.1]heptane, bicyclo[4.1.0]heptane, bicyclo[4.1.0]heptan-3-carboxylic acid, bicyclo[3.1.0]hexan-3-carboxylic acid, bicyclo[4.1.0]heptan-2-carboxylic acid, bicyclo[3.1.0]hexan-2-carboxylic acid, and bicyclo[2.2.1]heptan-2-carboxylic acid.

In accordance with yet some other examples, R₂ in A₃ARAM is selected from 2-cyclohexene and 3-cyclohexene.

Specific imidazoquinoline derivatives which can be used as allosteric modulators of the A₃AR are listed below:
N-(4-Methyl-phenyl)-2-cyclopentyl-1H-imidazo[4,5-c]quinolin-4-amine
N-(4-Methoxy-phenyl)-2-cyclopentyl-1H-imidazo[4,5-c]quinolin-4-amine
N-(3,4-Dichloro-phenyl)-2-cyclopentyl-1H-imidazo[4,5-c]quinolin-4-amine
N-(4-Chloro-phenyl)-2-cyclopentyl-1H-imidazo[4,5-c]quinolin-4-amine
N-(3-Methanol-phenyl)-2-cyclopentyl-1H-imidazo[4,5-c]quinolin-4-amine
N-([3,4-c]Indan)-2-cyclopentyl-1H-imidazo[4,5-c]quinolin-4-amine
N-(1H-indazol-6-yl)-2-cyclopentyl-1H-imidazo[4,5-c]quinolin-4-amine
N-(4-Methoxy-benzyl)-2-cyclopentyl-1H-imidazo[4,5-c]quinolin-4-amine
N-(1H-Indol-6-yl)-2-cyclopentyl-1H-imidazo[4,5-c]quinolin-4-amine
N-(Benzyl)-2-cyclopentyl-1H-imidazo[4,5-c]quinolin-4-amine
N-(Phenylethyl)-2-cyclopentyl-1H-imidazo[4,5-c]quinolin-4-amine
N-(3,4-Dichloro-phenyl)-2-cycloheptyl-1H-imidazo[4,5-c]quinolin-4-amine
N-(3,4-Dichloro-phenyl)-2-furyl-1H-imidazo[4,5-c]quinolin-4-amine
N-(3,4-Dichloro-phenyl)-2-cyclobutyl-1H-imidazo[4,5-c]quinolin-4-amine
N-(3,4-Dichloro-phenyl)-2-cyclohexyl-1H-imidazo[4,5-c]quinolin-4-amine
N-(3,4-Dichloro-phenyl)-2-1H-imidazo[4,5-c]quinolin-4-amine
N-(3,4-Dichloro-phenyl)-2-pentyl-1H-imidazo [4,5-c]quinolin-4-amine.

The above imidazoquinoline derivatives are regarded as allosteric modulators as they were shown to have, on the one hand, reduced affinity, if any, to the orthosteric binding sites of the A₁ and A_{2A}, A_{2B} adenosine receptors and reduced affinity to the orthosteric binding site of the A₃ adenosine receptor, and on the other hand, high affinity to the allosteric site of the A₃ adenosine receptor [International Patent Application No. WO07/089507, incorporated herein by reference].

A specifically preferred imidazoquinoline derivative in accordance with the present disclosure is N-(3,4-Dichloro-phenyl)-2-cyclohexyl-1H-imidazo[4,5-c]quinolin-4-amine (also referred to at times by the abbreviation LUF6000 or CF602), being an A₃AR allosteric modulator.

In the context of the general formulae disclosed herein, the following meaning for the various terms is to be considered:

The term “alkyl” is used herein to refer to a linear or branched hydrocarbon chain having from 1 to 10 carbon atoms and more preferably 1 to 6 carbon atoms including, but not limited to, methyl, ethyl, n-propyl, isopropyl, n-butyl, t-butyl, n-heptyl, octyl and the like.

Similarly, the terms “alkenyl” and “alkynyl” denote a linear or branched hydrocarbon chain having, respectively, from 2 to 10, or from 3 to 10 carbon atoms and more preferably 2 to 6 or 3 to 6 carbon atoms, the alkenyl or alkynyl having at least one unsaturated bond.

The alkyl, alkenyl or alkynyl substituents may be substituted with a heteroatom containing group. Thus, it should be understood that while not explicitly stated, any of the alkyl modifications defined hereinabove and below, such as alkylthio, alkoxy, akanol, alkylamine etc, also include the corresponding alkenyl or alkynyl modifications, such as, akenylthio, akenyloxy, alkenol, alkenylamine, or respectively, akynylthio, alkynyloxy, alkynol, alkynylamine.

The term “aryl” denotes an unsaturated aromatic carbocyclic group of from 5 to 14 carbon atoms having a single ring (e. g., phenyl) or multiple condensed rings (e. g., naphthyl or anthryl). Preferred aryls include phenyl, indanyl, benzimidazole.

The term “alkaryl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl and the like.

The term “Substituted aryl” refers to an aromatic moiety which is substituted with from 1 to 3 substituents as defined above. A variety of substituents are possible, as appreciated by those versed in the art. Nonetheless, some preferred substituents include, without being limited thereto, halogen, (substituted) amino, nitro, cyano, alkyl, alkoxy, acyloxy or alkanol, sulphonyl, sulphynyl.

The term “Halo” or “halogen” refers to fluoro, chloro, bromo and iodo, preferably to chloro.

The term “acyl” refers to the groups H—C(O)— as well as alkyl-C(O)—.

The term “alkanol” refers to the group —COH as well as alk-OH, “alk” denoting an alkylene, alkenylene or alkynylene chain.

The term “alkoxy” is used herein to mean —O-alkyl, including, but not limited to, methoxy, ethoxy, propoxy, isopropoxy, n-butoxy and the like.

The term “alkylthio” is used herein to mean —S-alkyl, including, but not limited to, methylthio, ethylthio, n-propylthio, isopropylthio, n-butylthio and the like.

The term “alkoxyalkyl” is used herein to mean -alkyl-O-alkyl, including, but not limited to, methoxymethyl, ethoxymethyl, n-propoxymethyl, isopropoxymethyl, n-butoxymethyl, isobutoxymethyl, t-butoxymethyl and the like.

The term “cycloalkyl” is used herein to mean cyclic hydrocarbon radicals including, but not limited to, cyclopropyl, cyclobutyl, cyclopentyl, cyclohexyl, cycloheptyl and the like.

The term “alkoxycarbonyl” is used herein to mean —C(O)O-alkyl, including, but not limited to, methoxycarbonyl, ethoxycarbonyl, propoxycarbonyl and the like.

The term “fused cycloalkyl” is used herein to mean any compound or substituent comprising at least two aliphatic rings which are connected at a single atom (to form a spirocyclic moiety), at two mutually bonded atoms or across a sequence of atoms (bridgehead). The fused rings may include any bicyclic, tricyclic as well as polycyclic moieties. Bicyclic substituents are preferred in accordance with some embodiments of the present disclosure.

The present disclosure also makes use of physiologically acceptable salts of an A₃AR selective ligand, such as the above-described compounds. A “physiologically acceptable salt” refers to any non-toxic alkali metal, alkaline earth metal, and ammonium salt commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the ligand with a suitable organic or inorganic acid. The acid addition salts are those which retain the biological effectiveness and qualitative properties of the free bases, and which are not toxic or otherwise undesirable. Examples include, inter alia, acids derived from mineral acids, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g., p-toluenesulphonic, acids.

The A₃AR ligand can be administered in a single dose (one time medication) or as a continuous treatment. In one example, the A₃AR ligand is used for long term treatment.

In the context of the present disclosure, long term treatment is to be understood to encompass a treatment window lasting for at least a period of days, weeks, months, or even years, until discontinuation for example due to intolerance, withdrawal of consent, or death. Though continuous, the treatment period may be divided into cycles (e.g., 4-week cycles) for the purpose of data recording. In an embodiment, the A₃AR ligand is administered every 12 h until discontinuation.

Further in the context of some examples of the present disclosure long term treatment encompasses chronic treatment, e.g., long term daily administration (once, twice, or thrice daily) at times even without an envisaged end point for the treatment. In some examples, the long-term treatment comprises at least one week of daily administration of the A₃AR ligand, at times, one-month daily treatment, at times, at least 2, 3, 4, 5, 6, or even 12 months of daily administration of the ligand, at times at least 1, 2, 3, 4, 5, 6 or more years of daily administration of the ligand.

In an embodiment, the treatment period is a long term treatment lasting for at least one year.

The A₃AR ligand is administered in amounts which are sufficient to achieve a therapeutic anti-cancer effect. As will be appreciated, the amount of the A₃AR ligand will depend on the severity of the disease, the intended therapeutic regimen, and the desired therapeutic dose. By way of example, where the dose is 50 mg per day and the desired administration regimen is 2 daily administrations, the amount of the active agent in the pharmaceutical composition will be 25 mg.

An amount effective to achieve the desired effect is determined by considerations known in the art. An “effective amount” for purposes herein must be effective to achieve a therapeutic effect, the therapeutic effect being as defined hereinbefore.

It is appreciated that the effective amount depends on a variety of factors including the affinity of the chosen A₃AR agonist to the A₃AR, its distribution profile within the body, a variety of pharmacological parameters such as half life in the body, on undesired side effects, if any, on factors such as age and gender of the subject to be treated, etc. The effective amount is typically tested in clinical studies having the aim of finding the effective dose range, the maximal tolerated dose, and the optimal dose. The manner of conducting such clinical studies is well known to a person versed in the art of clinical development.

In accordance with one embodiment of the invention, the administration of A₃AR agonist is preferably daily administration, between once and a few times a day, preferably once or twice a day, the dose in each administration being in the range of between about 1 to about 1000 μg/kg body weight, preferably 50 μg/kg-10 mg/kg body weight, preferably 100 μg/kg-5 mg/Kg body weight, or 200 μg/kg-1 mg/Kg body weight.

In some embodiments, the A₃AR ligand is Cl-IB-MECA administered orally in a dose of 1-50 mg, preferably 5-30 mg twice daily.

In a specific embodiment, the advanced solid tumor is advanced HCC, the A₃AR agonist is Cl-IB-MECA (namodenoson) and the dose is 25 mg given orally every 12 hours (i.e., twice a day).

The A₃AR agonist is formulated in a pharmaceutical composition. A “composition” in the context of the invention is intended to mean a combination of the active agent(s), together or separately, with a pharmaceutically acceptable carrier as well as other additives. The carrier may at times have the effect of improving the delivery or penetration of the active ingredient to the target tissue, improving the stability of the drug, slowing clearance rates, imparting slow-release properties, reducing undesired side effects etc. The carrier may also be a substance that stabilizes the formulation (e.g., a preservative). For examples of carriers, stabilizers, and adjuvants, see E. W. Martin, REMINGTON'S PHARMACEUTICAL SCIENCES, MacK Pub Co (June 1990).

The term “pharmaceutically acceptable carrier” in the context of the present invention denotes any one of inert, non-toxic materials, which do not react with the A₃AR agonist, and which can be added to formulations as diluents, carriers or to give form or consistency to the formulation.

The composition of the present invention is administered and dosed in accordance with good medical practice, taking into consideration the clinical condition of the individual patient, the site and method of administration, scheduling of administration, patient age, sex, body weight and other factors known to medical practitioners. The choice of carrier will be determined in part by the specific active ingredient, as well as by the specific method used to administer the composition. Accordingly, there is a wide variety of suitable pharmaceutical compositions of the present invention.

As noted above, the therapeutic use of an A₃AR agonist may at times be in combination with other anti-cancer drugs such monoclonal antibodies (e.g., the antibody atezolizumab alone or in combination with bevacizumab, or the VEGFR2 inhibitor ramucirumab, the anti-PD1 receptor monoclonal antibodies pembrolizumab and nivolumab, alone or in combination with the anti CTLA-4 antibody ipilimumab), and multi-kinase inhibitors (e.g., sorafenib, regorafenib, cabozantinib or lenvatinib). In such a combination treatment the other drug and the A₃AR agonist may be given to patients at the same time or at different times, depending on the dosing schedule of each of the drugs.

The invention has been described in an illustrative manner, and it is to be understood that the terminology which has been used, is intended to be a description rather than a limitation. Obviously, many modifications and variations of the present invention are possible in view of the above teaching. It is therefore, to be understood that within the scope of the appended claims, the invention may be practiced otherwise than as specifically described hereinafter.

### Exemplary Embodiments

### Study Participants

The study population consisted of patients aged ≥18 years with advanced stage/treatment-refractory HCC and CPB cirrhosis who did not tolerate sorafenib or whose disease had previously progressed on sorafenib treatment. The diagnosis of HCC in subjects without underlying cirrhosis at the time of diagnosis required cytology and/or histology; for subjects with underlying cirrhosis at the time of diagnosis, HCC diagnosis was established according to the American Association for the Study of Liver Diseases practice guidelines algorithm [Marrero J. A., et al., Hepatology. 2018; 68:723-750]. For subjects who had tolerated sorafenib, ≥3 weeks of prior treatment was required, terminating at ≥2 weeks before study entry. Inclusion criteria included: ECOG PS≤2; having CPB cirrhosis (i.e., CP score 7-9); aspartate aminotransferase (AST) and alanine aminotransferase (ALT) levels ≤5 times the upper limit of normal (ULN); total bilirubin, ≤3.0 mg/dL; serum albumin, ≥2.8 g/dL; prothrombin time (PT), ≤6 s longer than control; serum creatinine, ≤2.0 mg/dL; absolute neutrophil count, ≥1500×10⁹/L; and platelet count, ≥75,000×10⁹/L. Exclusion criteria included: the presence of hepatic encephalopathy; and gastrointestinal hemorrhage requiring transfusion occurring within the past 4 weeks.

### Study Design and Treatment

This was a multicenter, randomized, double-blind, placebo-controlled clinical trial (ClinicalTrials.gov identifier NCT02128958). The trial was conducted at 15 sites in Israel, Europe, and the United States.
Subjects were enrolled by the participating centers and were randomly assigned 2:1, using a central randomization schedule generated by an independent biostatistician with no stratification prior to randomization. Patients were randomized to either namodenoson (Cl-IB-MECA) 25 mg or matching placebo, administered orally every 12 hours until discontinuation due to intolerance, withdrawal of consent, or death. Though treatment was administered in a continuous manner, the treatment period was divided into 4-week cycles for the purpose of data recording. No crossover was initially allowed; however, by protocol amendment, patients that continued with blinded treatment were offered namodenoson (25 mg twice daily) upon unblinding of the treatment assignments. All study site personnel were blinded to patients' treatment throughout the study period.
The study was approved by all relevant national regulatory authorities and local Ethics Committees/Institutional Review Boards. The study was conducted in accordance with the Declaration of Helsinki, and written informed consent was obtained from all the patients.

### Assessments

The primary endpoint of the study was overall survival (OS). Secondary endpoints were progression-free survival (PFS), overall response rate (ORR), disease control rate (DCR), and safety. As in advanced HCC, PFS was found to be moderately correlated with OS; PFS is a reasonable secondary endpoint in this disease [Llovet J. M., et al., J. Hepatol. 2019, 70:1262-1277]. Disease response assessment was evaluated locally by 2 independent blinded radiologists using Response Evaluation Criteria in Solid Tumors (RECIST) version 1.1 [Eisenhauer E. A., et al. Eur. J. Cancer. 2009; 45:228-247]. Tumor status was assessed at baseline and every 8 weeks thereafter by computed tomography scan or magnetic resonance imaging. Safety was monitored through assessments of AEs using the US National Cancer Institute's Common Terminology Criteria for Adverse Events (CTCAE) v4.03. Changes from baseline in vital signs, clinical laboratory parameters, electrocardiograms, physical examinations, and ECOG PS were also assessed. AFP levels were assessed at baseline and every 4 weeks thereafter, as were laboratory parameters associated with hepatic dysfunction and cirrhosis, such as serum ALT, AST, bilirubin, and albumin levels, PT, and international normalized ratio. ALBI scores were calculated from the albumin and bilirubin levels, as previously described [Johnson P. J., et al. J. Clin. Oncol. 2015; 33:550-558].

### Biomarker Studies

Another secondary objective was to evaluate the relationship between white blood cell (WBC) A3AR expression (which has been suggested to mirror the expression in HCC tumor cells [Bar-Yehuda S., et al. Int. J. Oncol. 2008; 33:287-295]) as assessed at baseline and every cycle thereafter at selected study centers, (n=53 patients) and clinical response. A3AR mRNA expression in WBC was determined from blood collected to a PAXgene RNA tube (Qiagen, Venlo, The Netherlands), using the QuantiGene Plex 2.0© assay (Thermo Fisher, Waltham, Mass., USA). β-actin was used as a reference control, and the oligonucleotide probe sets were designed by Thermo Fisher. Luminescence from each specific probe set was captured by GloMax Multi (Promega, Madison, Wis., USA). A3AR was expressed in units, where 1 unit was defined as the mean of A3AR expression in healthy subjects (n=50). Healthy subjects were 20-70 years of age with no known illness and no prior treatment.

### Statistical Analysis

Power calculation determined that 75 deaths, assuming a hazard ratio of 0.5, would provide 80% power for the log-rank test at a significance level of 0.05. Primary efficacy analyses were performed on the ITT population. Descriptive statistics were used to summarize patient/tumor characteristics and safety. Kaplan-Meier curves were used to estimate OS/PFS, and between comparisons were performed using log-rank tests. The Cox proportional hazards regression model was used to assess the impact of covariates. ORR/DCR were determined using the normal approximation to the binomial distribution by treatment. The statistical analysis plan was amended prior to unblinding to include subgroup analysis by CP score. All statistical tests were 2-sided, and p<0.05 was considered statistically significant. Statistical analyses were conducted using SAS 9.4 (SAS Institute Inc., Cary, N.C., USA).

### Example 1

In a phase II blinded, randomized, placebo-controlled study, the namodenoson dose evaluated was oral 25 mg BID (twice daily) and the population consisted of 78 patients with advanced HCC and CPB cirrhosis who received namodenoson as second-line therapy. Patients were randomized 2:1 to receive namodenoson 25 mg BID (n=50) or placebo (n=28).

No treatment-related deaths were reported. Also, no patients withdrew from the study and no dose reductions were attributable to namodenoson. Importantly, no hepatotoxicity was reported and liver function tests demonstrated no adverse namodenoson-related effect. Mean serum albumin levels and albumin-bilirubin (ALBI) scores also did not change significantly in both arms throughout the study. Only one grade 3 treatment-related AE was reported (hyponatremia).

Analysis of Initial Results

With respect to anti-tumor efficacy, the primary trial endpoint of OS superiority over placebo was not met; median OS was 4.1 and 4.3 months for namodenoson and placebo, respectively (hazard ratio [HR], 0.82; 95% confidence interval [CI] 0.49-1.38; p=0.46). Similarly, there was no superiority with respect to progression-free survival (PFS). Within the CBP patients included in the study, the patients with Child-Pugh score 7 (the least severe form of hepatic dysfunction within the CPB category) were the largest group (34 patients in the namodenoson arm and 22 patients in the placebo arm). Preplanned analysis in which patients were evaluated by Child-Pugh subgroups demonstrated nonsignificant differences in OS and PFS for patients with Child-Pugh score 7. In this subcategory, the median OS was 6.9 in the namodenoson group vs 4.3 months in the placebo group (HR, 0.81; 95% CI 0.45-1.43; p=0.46). The median PFS was 3.5 months vs 1.9 months (HR, 0.89; 95% CI 0.51-1.55; p=0.67). In patients with Child-Pugh score 8 (13 patients; 7 namodenoson-treated and 6 placebo-treated), OS and PFS were similar between the namodenoson and placebo arms and were overall shorter than those reported for the subgroup of patients with Child-Pugh score 7 (OS: 3.3 vs 3.4 months, for the namodenoson and placebo arms respectively; HR, 0.88; 95% CI 0.28-2.77, p=0.83. PFS: 2.1 vs 1.9 months, respectively; HR, 0.71; 95% CI 0.23-2.17, p=0.53). The median OS and PFS values for the 9 patients with Child-Pugh Score 9, who were all in the namodenoson arm, were 3.5 and 2.2 months, respectively, similar to those in patients with Child-Pugh score 8. Exploratory analysis comparing OS by treatment arm and stratification by gender, alfa-fetoprotein levels, Eastern Cooperative Oncology Group (ECOG) Performance Status (PS), HPB, and HPC status, locoregional therapy, extrahepatic spread status, and portal vein thrombosis status found no statistically significant differences between the study arms in any of the subgroups, which could be attributed, in part, to the relatively small sample size in some of the groups.

Analysis of Long-Term Treatment

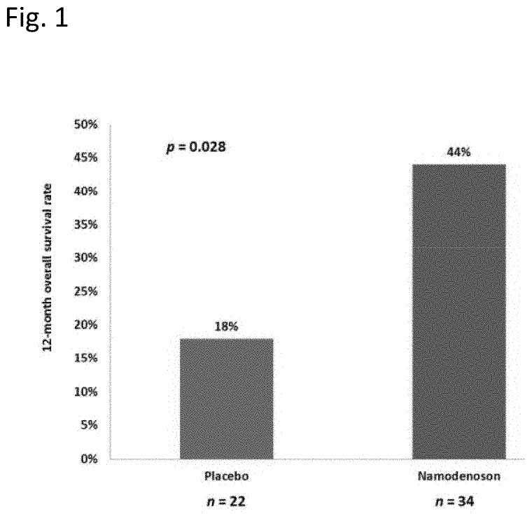

However, in contrast with the initial results, the difference in the 12-month OS rate was statistically significant (44% and 18% in the namodenoson and placebo arms, respectively; p=0.028) (FIG. 1).

Analysis of the response in all patients for whom at least 1 post-baseline assessment was available (55 patients; 34 namodenoson-treated and 21 placebo-treated) revealed that one patient in the namodenson group experienced CR, and that PR was achieved by 3 patients (9%) in the namodenson group vs none in the placebo group (Table 1). In the 3 patients who experienced PR, the duration of response was 2, 6, and 26 months.

TABLE 1
Best Observed Responses (RECIST 1.1) by Treatment Arm

| Response, n (%) | Namodenoson n = 34 | Placebo n = 21 |
|---|---|---|
| CR | 1 (2.94%) | 0 (0.0%) |
| PR | 3 (8.8%) | 0 (0.0%) |
| SD | 17 (50.0%) | 10 (47.6%) |
| PD | 14 (41.2%) | 11 (52.4%) |

As shown in Table 1 above one patient showed a complete response. This patient was treated for 5 years under the Open Label Extension program of the Phase II study of Namodenoson in the treatment of hepatocellular carcinoma (HCC) and experienced a Complete Response (CR) to namodenoson, meaning that all cancer lesions have cleared.

Under treatment with namodenoson (25 mg administered orally twice a day), the patient who had advanced metastatic HCC with underlying Child Pugh B7 (CPB7) cirrhosis has now survived five years. As indicated by a scan of the patients chest, abdomen, and pelvis the clinical benefits of treatment have included the disappearance of ascites, normal liver function, and the disappearance of peritoneal carcinomatosis leading to complete clearance of all cancer lesions.

1. A method of treating an advanced solid tumor said method comprising administering to a mammalian subject in need thereof an A₃ adenosine receptor (A₃AR) ligand or a pharmaceutical composition comprising said A₃AR ligand.

2. The method according to claim 1, wherein said advanced solid tumor is advanced hepatocellular carcinoma.

3. The method according to claim 2, for treating metastatic hepatocellular carcinoma.

4. The method according to claim 1, wherein said A₃AR ligand is an A₃AR agonist or an A₃AR allosteric modulator.

5. The method according to claim 4, wherein said A₃AR agonist is selected from the group consisting of N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA, namodenoson).

6. The method according to claim 4, wherein said A₃AR allosteric modulator is selected from the group consisting of:
N-(3,4-Dichloro-phenyl)-2-cyclopentyl-1H-imidazo[4,5-c]quinolin-4-amine;
N-(3,4-Dichloro-phenyl)-2-cycloheptyl-1H-imidazo[4,5-c]quinolin-4-amine;
N-(3,4-Dichloro-phenyl)-2-cyclobutyl-1H-imidazo[4,5-c]quinolin-4-amine; and
N-(3,4-Dichloro-phenyl)-2-cyclohexyl-1H-imidazo[4,5-c]quinolin-4-amine.

7. The method according to claim 1, wherein said method further comprises administration of an additional therapeutic agent.

8. The method according to claim 7 wherein said additional therapeutic agent is an anti-cancer drug, e.g., a monoclonal antibody and/or a multi-kinase inhibitor.

9. The method according to claim 2 wherein said subject has advanced hepatocellular carcinoma with Child-Pugh B (CPB) cirrhosis score of 7 (CPB7), Child-Pugh B (CPB) cirrhosis score of 8 (CPB8), or Child-Pugh B (CPB) cirrhosis score of 9 (CPB9).

10. The method according to claim 1 wherein said A₃AR ligand is administered once daily, twice daily, or thrice daily.

11. The method according to claim 1 wherein said A₃AR ligand is administered every 12 hours throughout the treatment period.

12. The method according to claim 11 wherein said A₃AR ligand is administered in a continuous manner.

13. The method according to claim 1 wherein said A₃AR ligand is administered at an amount of 50 μg/kg-10 mg/kg body weight, preferably 100 μg/kg-5 mg/Kg body weight, or 200 μg/kg-1 mg/Kg body weight.

14. The method according to claim 1 wherein said A₃AR ligand is Cl-IB-MECA and wherein said Cl-IB-MECA is administered orally in a dose of 1-50 mg, preferably 5-30 mg twice daily.

15. A method of increasing overall survival of subjects with advanced hepatocellular carcinoma (HCC) and a CPB7 score, said method comprises administering an A₃AR ligand (e.g., Cl-IB-MECA) to said subject.

16. The method according to claim 15, wherein said increase in overall survival is measured after a treatment period of 9 months, 10 months, 12 months or more, and wherein said treatment comprises administration of the A₃AR ligand (e.g., Cl-IB-MECA) orally in a dose of 1-50 mg, preferably 5-30 mg twice daily.

17. The method according to claim 15 wherein said subject received the A₃AR ligand as a second-line therapy.

18. The method according to claim 15 wherein said administration is for a treatment period of at least 9 months, at least 10 months, at least one year, at least 2 years, at least 3 years, at least 4 years or at least 5 years.

19. A pharmaceutical composition comprising an A₃AR ligand (e.g., Cl-IB-MECA), and a pharmaceutically acceptable carrier or diluent wherein said pharmaceutical composition is for increasing overall survival of subjects with advanced HCC and a CPB7 score.

20. The pharmaceutical composition according to claim 19, wherein said increase in overall survival is measured after a treatment period of 12 months or more, and wherein said treatment comprises administration of said A₃AR ligand (e.g., Cl-IB-MECA) orally in a dose of 1-50 mg, preferably 5-30 mg twice daily.
